(19) United States
(12) Patent Application Publication
(10) Pub. No.: US 2023/0397680 A1
(43) Pub. Date: Dec. 14, 2023

(54) MANUAL SAMPLING DEVICE AND METHODS

(71) Applicant: Fremonta Corporation, San Jose, CA (US)

(72) Inventors: Yongqing Huang, San Jose, CA (US); Wei Wu, San Jose, CA (US); Florence Wu, Fremont, CA (US); Garth Hoffmann, San Jose, CA (US); Eric Wilhelmsen, San Jose, CA (US)

(21) Appl. No.: 18/332,199

(22) Filed: Jun. 9, 2023

Related U.S. Application Data
(60) Provisional application No. 63/350,782, filed on Jun. 9, 2022.
(60) Provisional application No. 63/485,690, filed on Feb. 17, 2023.

(51) Int. Cl. A41D 19/01

(52) U.S. Cl. CPC A41D 19/01

(57) ABSTRACT

Manual samplers and sampling methods that facilitate improved dexterity and ease of use and that can be manufacture quickly and cost effectively are provided herein. Such sampler can include sampling sheet(s) with a pocket defined by seams to accommodate a user's entire hand for manual sampling of one or more products, in particular aggregate sampling of multiple products. Samplers can include entry seams to fit along the user's writs to retain the sampler on the user's hand and/or interior seams that engage the user's digits to improve dexterity while contact sampling of uneven surfaces.

### CROSS REFERENCE TO RELATED APPLICATIONS

This application is a Non-Provisional of and claims the benefit of priority to U.S. Provisional Application No. 63/350,782 filed Jun. 9, 2022 and U.S. Provisional Application No. 63/485,690 filed Feb. 17, 2023, the entire contents of which are incorporated herein by reference.

### BACKGROUND

### Field of the Invention

The present invention is directed to improved sampling devices and method for sampling, especially aggregate sampling of food products and environmental sampling.

### Description of the Related Art

Recent developments in aggregated sampling teach the utility of pocketed samplers. Despite these advancements, users of these samplers find that hand fatigue and poor fit reduce functionality. Moreover, manual sampling can be difficult given the uneven, often wet surfaces being sampled. While sampling gloves have also been proposed, such sampling gloves are costly to manufacture and may also require multiple sizes to accommodate differing hand sizes. Therefore, improved samplers are needed that address these deficiencies and that are more cost-effective.

### SUMMARY

In one aspect, the invention pertains to a sampler having a pocket that accommodates a user's hand and a section with one or more interior seams (e.g. weld lines or stitching that extend within the interior of the pocket). These interior seams engage the user's fingers and/or wrist to retain the sampler on the user's hand during sampling irrespective of hand size. In some embodiments, the one or more interior seams are parallel with the lengthwise edges or form acute angles relative a first seam so as to provide the desired prevention of hand fatigue and retention on the users hand. The samplers can be formed of one or more sheets of material that are bound by the seams to form the pocket. The seams can be weld lines, stitching, adhesive or any suitable type of bond. In some embodiments, the samplers can be formed to receive the user's entire hand, as a mitt, which is well suited for contact sampling of products, particularly aggregate sampling of food products. In other embodiments, the sampler is sized to receive only the fingers or finger tips, for example, up the fingers up to a portion of the palm and not including the user's thumb, which is better suited for light sampling used for environmental sampling, which may be performed in various zones of a meat or food processing facility, or various other locations, such as airports, shipping and commercial facilities. In any application, the sampler can further include interior seams that define regions that receive the user's fingers and are sized and arranged to accommodate a range of user hand sizes and different wearing techniques per the user's preference. Accordingly, such samplers can provide a one-size-fits all sampler that can accommodate most any user's hand size and still provide retention on the user's hand or fingers during manual contact sampling.

### BRIEF DESCRIPTION OF THE DRAWINGS

FIGS. 1-4 depict examples of mitt samplers configured for placement over the user's hand for sampling along or near the open face of the hand during sampling of surfaces or products with the user's open hand, in accordance with some embodiments.

FIG. 5 depicts a conventional manual sampling approach utilizing a standard sampling sheet to illustrate the drawbacks of the conventional approach.

FIGS. 6-9 depict the mitt samplers of FIG. 1-4 as worn on a user's hand, in accordance with some embodiments.

FIGS. 10A-10B depict fingertip samplers suitable for environmental sampling, in accordance with some embodiments.

FIG. 11 depicts a prototype example of a finger-tip sampler worn on the digits of the user for sampling primarily with the fingertips during sampling, in accordance with some embodiments.

### DETAILED DESCRIPTION

Disclosed herein are a specific class of improvements to manual sampling devices suitable for use with aggregated sampling, such as the methods taught in U.S. Pat. Nos. 10,663,446 and 10,935,535, the entire disclosures of which are incorporated herein by reference. It is appreciated however, that the improved manual samplers described herein can be used for any type of sampling, including non-aggregate sampling, and various other methods of aggregate sampling.

### Aggregate Sampling

While the food safety industry has utilized various approaches to sampling of food products, in recent years, aggregate sampling methods have been developed that provide marked advantages over conventional destructive sampling methods. In aggregate sampling, an absorptive sampling medium contacts the surface of multiple products to obtain an aggregate sample that is representative of the entire batch, lot or sub-lot of products. The sampling medium can contact all, a majority or only some of the products. In other embodiment, the aggregate sample can include multiple samples obtained from differing locations on a single carcass or half-carcass. In some embodiments, the sampling medium is a sheet of material, such as a cloth that is manually manipulated by the user across a surface of the products being sampled to obtain a contact sample with the cloth. Such sampling methods have gained widespread acceptance for sampling of food products, particularly animal meat products (e.g. beef trim, carcasses, poultry, fish, lamb, etc.) to determine the presence of pathogens (e.g. Salmonella, Listeria, pathogenic E. coli, etc.) Some applications utilize manual sampling to obtain the aggregate sample. However, there are difficulties associated with this approach. Typically, the surfaces of these products are uneven and often wet such that manually rubbing such products with a sampling sheet can be difficult and a user may regularly lose grasp of the sampling sheet. During sampling of a large number of food products, this repeated grasping of the sampling sheet can induce fatigue and discomfort in the user's hands, thereby limiting the user's dexterity and reducing their ability to sample products. This can be true not only for food products, but for various other products as well, including products in commerce or transit (e.g. airport luggage, shipped goods, etc.).

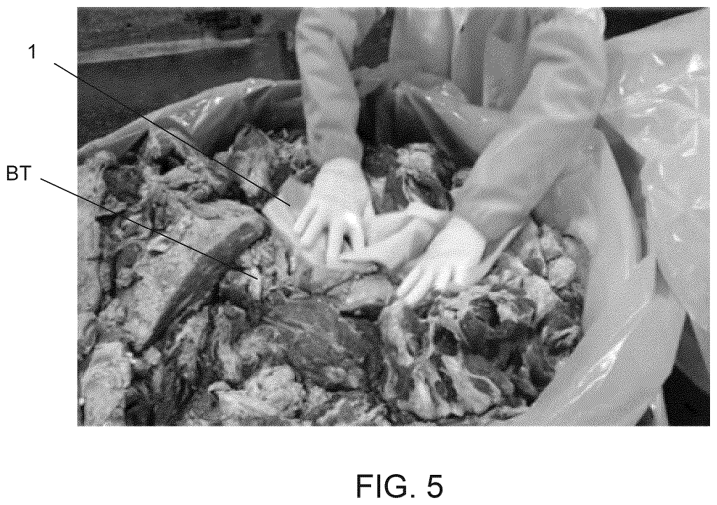

In safety sampling of food products, for example beef trim or carcasses, poultry, lamb and fish, the user obtains an aggregate sample of a group (e.g. batch, combo-bin, lot or sub-lot) of the food product. The user is instructed to briskly rub the exposed surface of the food product being sampled to ensure that particles and/or fluid residue adhere to or are absorbed by the sampling medium. Typically, the surface being sampled is highly irregular, which is true for beef trim, beef carcasses, poultry, lamb and fish. In a conventional approach, depicted in FIG. 5, the user unfolds a single sampling sheet, spreads it across the surface and forcefully rubs the sampling sheet 1 across the surface of the beef trim BT. Given that the surface is irregular and often wet or tacky, the sampling sheet is difficult to grasp and often requires the user to repeatedly grasp and reposition the sampling sheet while rubbing, which is a laborious task such that dexterity is compromised, and the user is prone to fatigue. Therefore, in order to improve dexterity of the user during sampling, the invention provides a mitt-type sampling device having a pocket sized to receive the entire hand of the user and is retained on the user's hand during sampling, as detailed further below.

### Mitt Sampling Devices

Pocket samplers that substantially fit the entire hand of the user can be considered “mitt samplers.” The following disclosure describes various features that can be utilized in “mitt samplers” but can also potentially be incorporated into other types of pockets sampler, including samplers having pockets meant for receiving a tool or equipment, or fingertip samplers described further below.

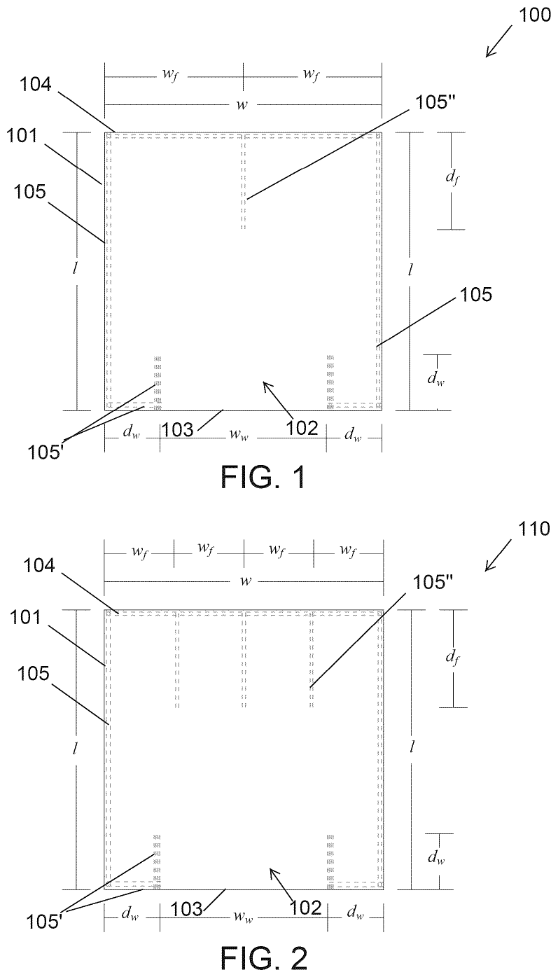

Examples of mitt samplers are shown in FIGS. 1-4. While certain dimensions and aspect ratios are shown here, it is appreciated that one or more of these dimensions or proportions could be modified or additional seams could be removed or added and still be in accordance with the concepts described herein.

Although pocket sampling devices are functional, their use is hindered by the need for multiple sizes and or mechanisms for constraining the opening for retention on the hand of the user. Changing the size of the opening for the wrist is functional but would typically require inventory and managing multiple sizes which add costs throughout the system which are ultimately passed on to the consumers. Adding a draw string or ribbon has the potential for becoming a foreign object and adds an additional step for the user. Adding elastic to the edge such as for a hairnet adds material to the sampler that is not approved for food contact. None of the traditional options adequately addressed the needs.

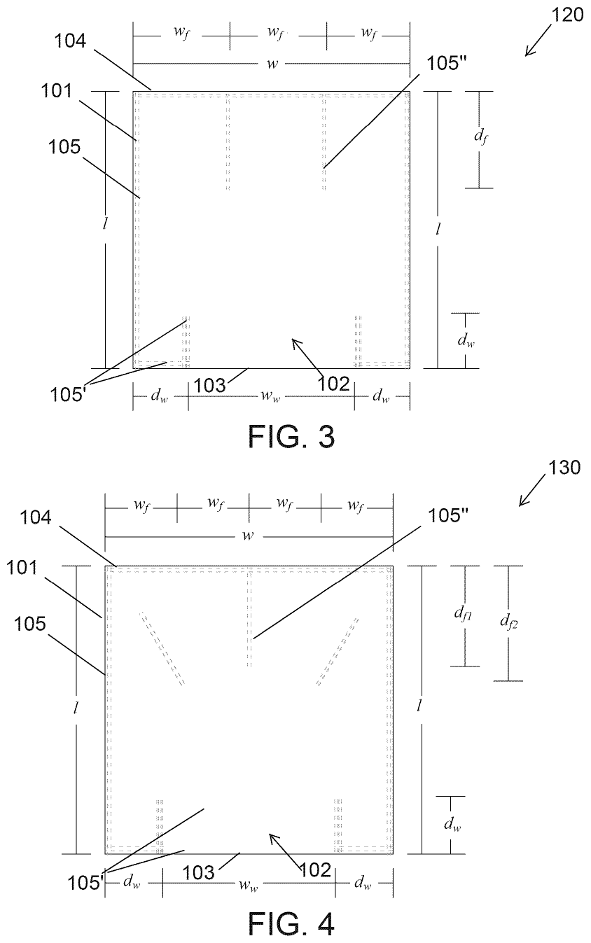

One advancement realized by the present invention was that providing passive internal resistance to sliding on the user's hand was sufficient to enable easy use of the pocketed sampling device. Gloves were considered but again multiple sizes would be required and their manufacture is more complex and approaches being cost-prohibitive as the sampler is a single-use, disposable product. One realization was that a partial approach to the glove configuration yielded a more preferred option. Three lines of sonic welding as illustrated in any of FIGS. 2 and 4 realized this concept. Further research showed that even a single line of welding, as shown in FIG. 1, or a pair of line welds, as shown in FIG. 3, can also met this need and sufficiently engages the digits of the user's hand in order to retain the sampler on the user's hand and facilitate sampling.

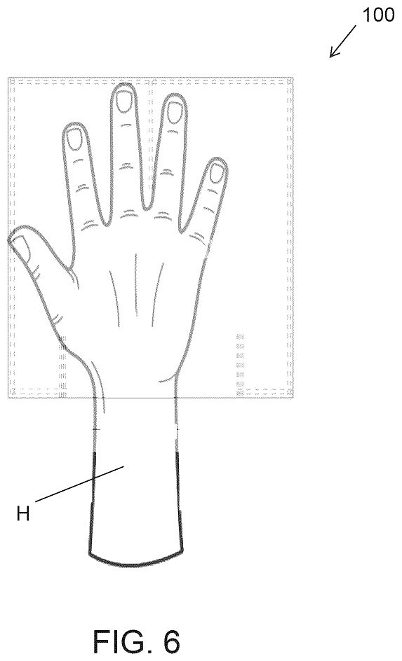

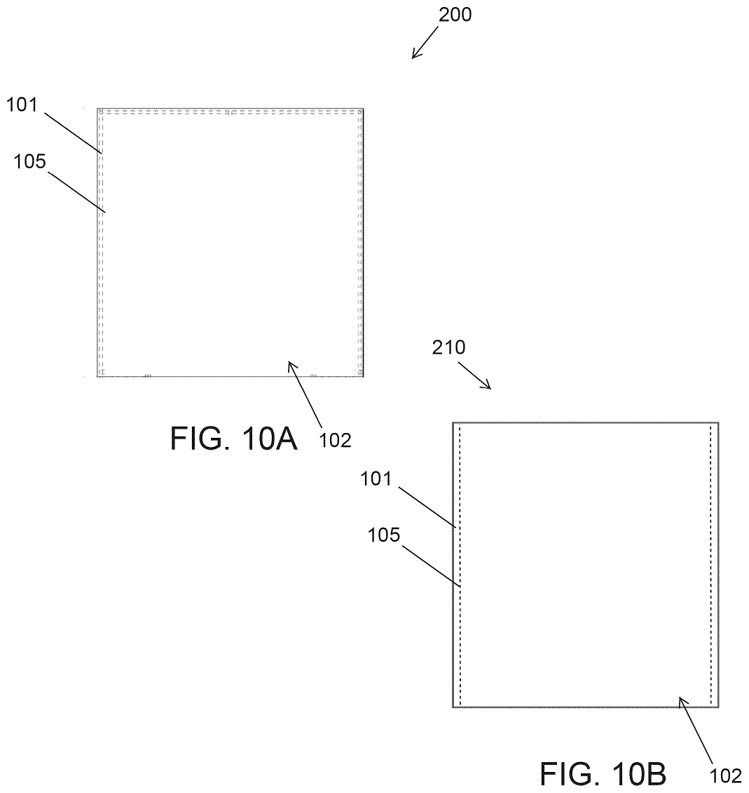

FIG. 1 shows sampler 100 which includes sampling sheet(s) 101 with a pocket 102 formed by seams 105 along select edges to define the pocket. It is appreciated that the sampler could be formed by two sampling sheets overlaid, as shown, or by a single sampling sheet folded in half (as shown in FIG. 10B, which would require seams on the lateral sides). In this exemplary embodiment, the overall width is about 10″ and the overall length is about 10.″ Additional entry seams 105′ can be included near the opening 103 of the pocket 102 to reduce the size of the opening and better engage a wrist of the user to retain the sampler on the user's hand during sampling. In this embodiment, the entry a extend inward about 2.5″ along the bottom edge and inward toward the center by about 2.5″although any suitable dimension could be used. In this embodiment, the sampler includes a single seam 105″extending from the distal edge (parallel with the lateral sides) within the interior of the pocket so as to extend between the digits of the user's hand to facilitate manual rubbing of the sampler against the surface being sampled. In this embodiment, the single seam extends from a distal edge 104 by a distance d_{f}, about 3.5″ inches, although any suitable distance could be used. In this embodiment, the single interior seam extends from a distance w_{f} (half) the width. It is appreciated that these exact dimensions are exemplary, and variations could be used. FIG. 6 shows sampler 100 when worn by a user's hand H, which better illustrates engagement of the user's digits with the interior seams and the entry seams with the user's wrist.

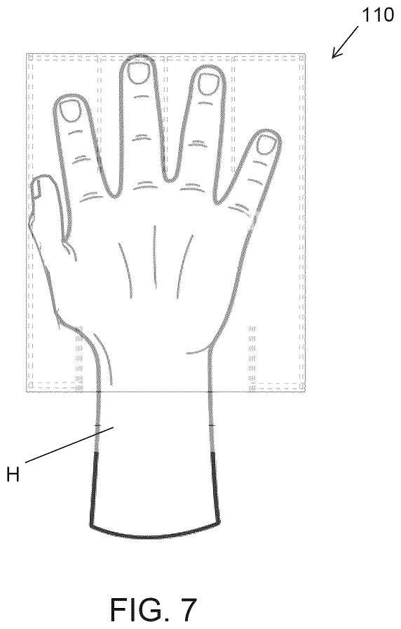

FIG. 2 shows sampler 110 which includes sampling sheet(s) 101 with a pocket 102 formed by seams 103 along select edges. It is appreciated that the sampler could be formed by two sampling sheets overlaid, or by a single sampling sheet folded in half (which would require only seams on the lateral sides). In this exemplary embodiment, the overall width is about 10″ and the overall length is about 10.″ Additional entry seams 105′ can be included near the opening 103 of the pocket 102 to reduce the size of the opening and better engage a wrist of the user to retain the sampler on the user's hand during sampling. In this embodiment, the entry seams extend inward about 2.5″ along the bottom edge and inward by about 2.5″, although any suitable dimension could be used. In this embodiment, the sampler includes three interior seams 105″ extending from the distal edge 104 to the interior (parallel with the lateral sides) defining four regions for each of the user's digits during sampling. In this embodiment, the three interior seams extend a distance d_{f}, about 3.5″ inches, although any suitable distance could be used. In this embodiment, the three interior seams are distributed at a regular interval, w_{f}, a portion of the width, for example 2.5″ for a 10″ square sampler. It is appreciated these exact dimensions are exemplary, and variations could be used. FIG. 7 shows sampler 110 when worn by a user's hand H, which better illustrates engagement of the user's digits with the interior seams and the entry seams with the user's wrist.

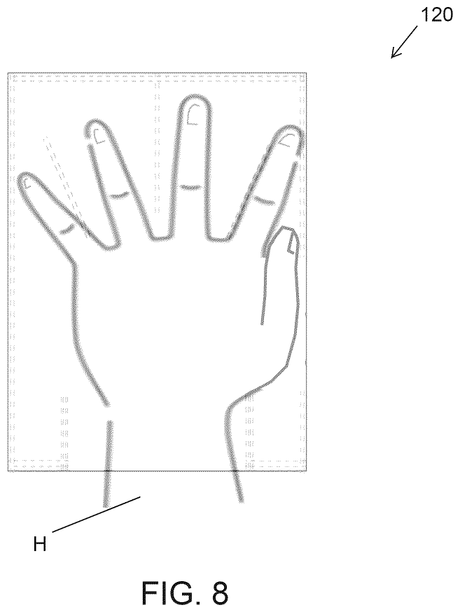

FIG. 3 shows sampler 120 which includes a sampling sheet(s) 101 with a pocket 102 formed by seams 105 along select edges. It is appreciated that the sampler could be formed by two sampling sheets overlaid, or by a single sampling sheet folded in half (which would require only seams on the lateral sides). In this exemplary embodiment, the overall width is about 10″ and the overall length is about 10.″ Additional entry seams 105′ can be included near the opening 103 of the pocket 102 to reduce the size of the opening and better engage a wrist of the user to retain the sampler on the user's hand during sampling. In this embodiment, the entry seams extend inward about 2.5″ along the bottom edge and inward by about 2.5″, although any suitable dimension could be used. In this embodiment, the sampler includes two interior seams 105″ extending from the distal edge 104 (parallel with the lateral sides) within the interior of the pocket defining three regions for the user's digits during sampling. In this embodiment, the two interior seams extend a distance of about 3.5″ inches, although any suitable distance could be used. In this embodiment, the three interior seams are distributed by a regular interval of w_{f} the width, 3.3″ for a 10″ square sampler. It is appreciated that these exact dimensions are exemplary, and variations could be used. FIG. 8 shows sampler 120 when worn by a user's hand H, which better illustrates engagement of the user's digits with the interior seams and the entry seams with the user's wrist.

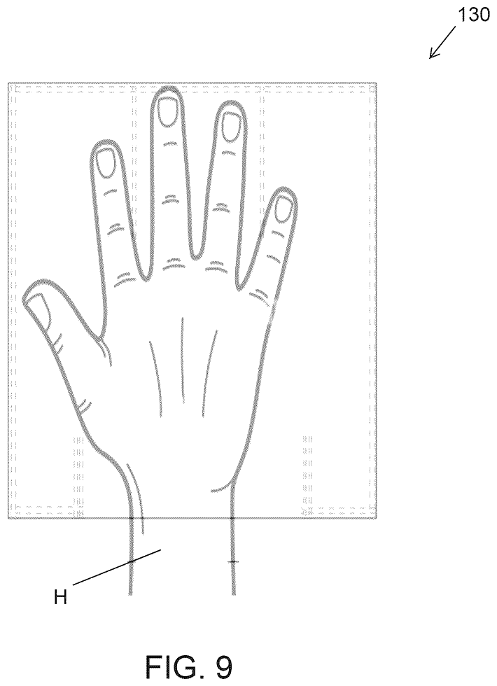

FIG. 4 shows sampler 130 which includes sampling sheet(s) 101 with a pocket 132 formed by seams 105 along select edges to form the pocket. It is appreciated that the sampler could be formed by two sampling sheets overlaid, or by a single sampling sheet folded in half (which would require only seams on the lateral sides, as shown in FIG. 10B for example). In this exemplary embodiment, the overall width is about 10″ and the overall length is about 10.″ Additional entry seams 105′ can be included near the opening 103 of the pocket 102 to reduce the size of the opening and better engage a wrist of the user to retain the sampler on the user's hand during sampling. In this embodiment, the entry seams extend inward about 2.5″ along the bottom edge and inward by about 2.5″, although any suitable dimension could be used. In this embodiment, the sampler includes three interior seams 105″ extending from the distal edge 104 to the interior defining four regions for the user's fingers, the seams extending radially toward a central region so as to correspond to the user's fingers when splayed outward, which further facilitate manual rubbing of the sampler against the surface being sample by the user's open hand. In this embodiment, the two interior seams extend a distance, d_{f}, about 3.5″ inches for a 10″×10″ sampler, although any suitable distance could be used. In this embodiment, the three interior seams are distributed at a regular interval, w_{f}, the width, 3.3″ for a 10″ square sampler. It is appreciated that these exact dimensions are exemplary, and variations could be used. FIG. 9 shows sampler 130 when worn by a user's hand H, which better illustrates engagement of the user's digits with the interior seams and the entry seams with the user's wrist.

These designs could include various modifications, including adding a second line of welding that is parallel or forms an acute angle with another line. It is only potentially needed to inexpensively add sufficient internal resistance to allow easy retention of the sampler on the hand of the user without forcing the need for multiple sizes. In one aspect, the one size fits all design can be an important cost saving measure that benefits manufacturers, users and ultimately the consumer.

On a practical note, the concept has been implemented in terms of sonic welding which would be the preferred mode of manufacture for samplers made of non-woven polyolefin material. However, stitching or adhesives or any suitable bonding approach made be more applicable for some applications and some materials. These selections can be driven by purpose or manufacturer preference. While a non-woven polyolefin material is preferred, it is appreciated that the sampling sheets can be formed of any material suitable for sampling.

Clearly, the partially split pockets samplers described here are a marked advancement to the sampling systems described for aggregated sampling of many food products as taught in U.S. Pat. Nos. 10,663,446 and 10,935,535, each of which are incorporated by reference in their entirety for all purposes.

It is appreciated that variations in embodiments can be realized to achieve different effects and benefits. In some embodiments, the grasping feature is inside the pocket is a feature that allows sampler retention is one marked advantage. In a preferred embodiment, there is a single line of welding. In some embodiments, multiple weld lines can provide the same or similar function as gloves, but can be configured and placed so as to accommodate differing of sizes of hands, unlike gloves. In some embodiments, weld lines can be widened by second line of welding which need not be parallel. For example, a narrow “V” arrangement of weld lines would function well.

### Environmental Sampling

Another sampling application that can be improved upon by the sampler and method herein is environmental sampling, which typically entails sampling of a surface in a given environment (e.g. food processing facility, warehouse, airport, laboratory, etc.). Typically, the sampling protocol for environmental sampling involves contact surface sampling of a surface, which could be an exposed surface of a processing machine, countertop, wall, floor etc. Typically, environmental sampling is performed according to a sampling protocol to determine whether a given environmental is contaminated. The sampling protocol often divides the environment into zones based on proximity and likelihood of transfer of any contamination into the product line. For example, zones can include: Zone 1—Food contact surface (e.g. conveyors, pipe chutes that food flows over); Zone 2—areas that could migrate contamination into zone 1, (e.g. over lines/adjacent surfaces, such as next to conveyor); Zone 3—adjacent area that are not likely (e.g. walls/drains); and Zone 4—areas just outside of Zone 3, where traffic might carry into contamination. Generally, environmental samples are obtained for each zone so that the presence of contamination can be determined separately for each zone. In some applications, aggregate samples are obtained from multiple surfaces within a given zone. It is advantageous to utilize a similar sampling device as described herein that facilitates environmental sampling and allows a user to readily collect an aggregate sample from multiple surfaces with a sampling sheet with greater ease and efficiency. Accordingly, a modification of the mitt sampling approach above is to configure a smaller pocket sampler that is specifically configured and sized to receive the user's fingertips. In some embodiments, the sampler pocket is dimensioned to receive all four digits and optionally part of the upper palm. Since environmental sampling requires less force/dexterity during sampling, the sampler can omit the interior seams that engage the fingers, previously described. Thus, the environmental sampler, also referred to as a fingertip sampler, can be one or more sheets with one or more seams that define a smaller pocket to fittingly receive the user's digits to facilitate environmental sampling. The sampler also does not require any addition entry seams as the opening is sized to fit the four digits and upper hand.

### Fingertip Sampling Devices

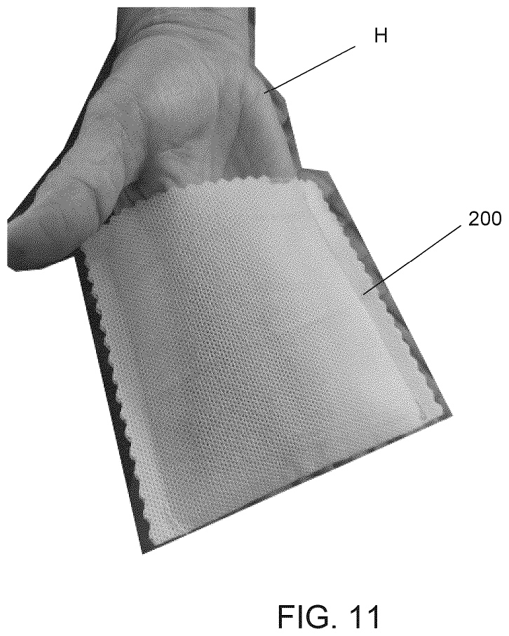

Pocket samplers can be configured in a wide variety of sizes and shapes to better meet sampling needs. One particular configuration, the environmental or fingertip sampler, meets the particular set of needs of environmental sampling. These special functions are described further below, and an example of a finger tip sampler is shown in FIG. 10A-10B and an exemplary prototype is shown in FIG. 11. Although this particular prototype shows edges bound with a threaded material, it is appreciated that the edges may be bound by one or more weld lines, as described previously. Similarly although the edges are shown as scalloped, it is appreciated that the edges can be straight as in previous designs.

FIGS. 10A-10B show a fingertip sampling device is a pocket sampler sized to fit over just the four fingers of a hand, which allows the user to leverage the dexterity of the human hand to conform to surfaces. It can also be sized to easily sample known and or constant areas. A nominal 10 cm (4″) square dimension is of particular utility for sampling 100 cm² as is common for many enumeration-based sampling procedures. A single sampling or a sampling in two orthogonal dimensions provides a reproducible area without the need for a template or guide. FIG. 10A shows an environmental sampler 200 formed of sample sheets 101 with a pocket 102 defined by seams 105 along three edges. In this embodiment, the sampler 200 is formed of two overlaid sheets that are seamed together along three sides, leaving an opening to receive the user's fingers in the pocket. FIG. 10B shows an environmental sampler 200 formed of a sample sheet 201 with a pocket 202 defined by seams 203. In this embodiment, the sampler 200 is formed of a single sheet folded in half and seamed along opposite lateral sides. FIG. 11 shows the environmental sampler 200 when worn on the user's hand H for environmental sampling.

Microbial sampling generally falls into two categories, qualitative and quantitative. These categories overlap when examined on a fundamental level, when the details of the method are examined. Qualitative tests are generally presence versus absence tests. However, they are quantitative in that there is an inherent detection limit. Quantitative tests must have a basis. This basis can be per gram, per area, per unit. The fingertip sampler is most helpful for sampling per area. However, it can be used for qualitative tests with a defined detection limit.

A particular embodiment of the fingertip sampler is fabricated by folding a piece of fabric in half across the short dimension. The overlapping sides are sealed together by some means, suture threads, welds or any suitable type bond. With polyolefin fabric sonic welding is a preferred choice to form a pocket about 12 cm wide and 10 cm deep. The additional width allows fingers to be inserted and generates about a 10 cm surface. While certain dimensions are described here, it is appreciated that one or more of these dimensions could be modified or that weld lines could be removed or—added and still be in accordance with the concepts described herein.

While specific embodiment are described herein, it is appreciated that variations or alternative combinations of features are within the spirit and scope of the invention. As used herein, the term “about” is considered to mean +/−10% of the recited value.

What is claimed is:

1. A sampler comprising:
one or more sheets of a sampling material disposed atop one another or folded; and
one or more edge seams between engaged surfaces of the one or more sheets, wherein the one or more edge seams are disposed along or near one or more edges of the one or more sheets to define a pocket having an open side for receiving at least a part of a hand of a user to facilitate manual sampling of a surface.

2. The sampler of claim 1, wherein the pocket is sized so as to accommodate a user's entire hand up to the user's wrist, thereby comprising a mitt sampler.

3. The sampler of claim 1, further comprising:
one or more entry seams disposed along edges and/or surfaces adjacent or near the open side of the pocket to retain the sampler on the user's hand.

4. The sampler of claim 1, further comprising:
one or more interior seams extending from or near a distal edge of the sampler to an interior region of the pocket so that the one or more interior seams engage one or more digits of the user's hand during sampling.

5. The sampler of claim 4, wherein the one or more interior seams comprise a single seam extending in a lengthwise direction from at or near mid-point of the distal edge.

6. The sampler of claim 4, wherein the one or more interior seams comprise two or more seams extending in a lengthwise direction from at or near a mid-point of the distal edge and are distributed along regular intervals.

7. The sampler of claim 4, wherein the one or more interior seams comprise a plurality of seams extending radially from a central region to the distal edge.

8. The sampler of claim 1, wherein the one or more sheets is a single sheet folded over on itself.

9. The sampler of claim 8, wherein the single sheet is rectangular.

10. The sampler of claim 8, wherein after folding, the folded single sheet is about 10 inches by 10 inches.

11. The sampler of claim 1, wherein the one or more sheets comprise a non-woven polyolefin.

12. The sampler of claim 1, wherein the one or more seams comprise an ultrasonic weld.

13. The sampler of claim 12, wherein at least some of the one or more seams comprise a double ultrasonic weld.

14. The sampler of claim 1, wherein the one or more sheets comprise two sheets of same or similar size laid atop one another.

15. The sampler of claim 1, wherein the pocket sampler is square or rectangular.

16. The sampler of claim 1, wherein the sampler is sized so as to fit only four digits of the user's hand to facilitate environmental sampling.

17. The sampler of claim 1, wherein the pocket sampler is about 3-4 inches in width by 3-4 inches in length.

18. A method of sampling comprising:
inserting a user's hand into a pocket of a pocket sampler as in claim 1;
obtaining a sample by contacting a surface of a product or a zone being sampled with the sampler, wherein contacting comprises moving the sampler across the contact surface while applying a force suitable for contact sampling; and
removing the sampler from the user's hand for subsequent testing.

19. The method of claim 18, wherein the pocket is sized so as to accommodate a user's entire hand up to the user's wrist, thereby comprising a mitt sampler.

20. The method of claim 18, further comprising:
contacting multiple products with the sampler so as to obtain an aggregate sample of multiple products for pathogenic testing of a batch or lot of the products.

21. The method of claim 20, wherein the multiple products comprise at least some of a lot of products.

22. The method of claim 21, wherein the lot of products comprise food products.

23. The method of claim 2, wherein the food products comprise any of: beef, beef trim, beef carcass, poultry, lamb, and fish.

24. The method of claim 18, wherein the pocket is sized so as to accommodate only a user's digits and part of the palm, thereby comprising a fingertip sampler suited for environmental sampling.

25. The method of claim 24, wherein further comprising:
contacting multiple surfaces within a given zone to obtain an aggregate sample for environmental testing of the zone.
